(19) United States
(12) Patent Application Publication
(10) Pub. No.: US 2026/0263258 A1
(43) Pub. Date: Sep. 10, 2026

(54) FLUID COLLECTION DEVICES HAVING A FLUID PERMEABLE BODY WITH A HYDROPHILIC MATERIAL SECURED TO A NON-WOVEN MATERIAL, AND METHODS OF MANUFACTURE

(71) Applicant: PUREWICK CORPORATION, Covington, GA (US)

(72) Inventors: Zhihui Yin, Lilburn, GA (US); Michael Anderson, Monroe, GA (US)

(21) Appl. No.: 19/490,075

(22) PCT Filed: Jun. 8, 2023
(86) PCT No.: PCT/US2023/024805

§ 371 Date: Dec. 4, 2025

(51) Int. Cl. A61F 5/44

(52) U.S. Cl. CPC A61F 5/44

(57) ABSTRACT

Embodiments disclosed herein are methods of manufacture of fluid collection devices, and related systems and devices. A method of manufacturing a fluid collection device includes securing a first layer including fluid permeable non-woven material to a second layer with an adhesive to form a fluid permeable body. The second layer includes one or more of a bamboo material, a cotton material, and/or a non-woven hydrophilic material. The method of manufacturing the fluid collection device also includes positioning the fluid permeable body in a chamber at least partially defined by a fluid impermeable barrier such that the fluid permeable body extends at least partially across an opening of the fluid impermeable barrier.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, pressure ulcers spills, and other hygiene issues. Urinary catheters be can be uncomfortable, painful, and can cause urinary tract infections.

Thus, users and manufacturers of fluid collection devices continue to seek new and improved devices, systems, and methods to collect urine.

### SUMMARY

Embodiments disclosed herein include fluid collection devices, and related assemblies, systems, and methods of manufacture. In an embodiment, a method of manufacturing a fluid collection device is disclosed. The method includes securing a first layer including fluid permeable non-woven material to a second layer with an adhesive to form a fluid permeable body, the second layer including one or more of a bamboo material, a cotton material, and/or a non-woven hydrophilic material. The method includes positioning the fluid permeable body in a chamber at least partially defined by a fluid impermeable barrier such that the fluid permeable body extends at least partially across an opening of the fluid impermeable barrier.

In an embodiment, a method of manufacturing a fluid permeable member for a fluid collection device is disclosed. The method includes securing a first layer including fluid permeable non-woven material to a second layer to form a fluid permeable body, the second layer including one or more of a bamboo material, a cotton material, and/or a non-woven hydrophilic material. The method includes rolling the fluid permeable body around a mandrel such that two opposing edges of fluid permeable body are adjacent longitudinally along the mandrel and the fluid permeable body forms a generally cylindrical shape. The method includes securing a metal member to the mandrel with a distal end of the metal member securing to the mandrel proximate to a distal end of the fluid permeable body, a proximal end of the metal member proximate to a proximal end of the fluid permeable body, and an intermediate region of the metal member proximate to the two opposing edges of the fluid permeable body. The method includes thermally sealing at least the second layer of fluid permeable body at the two opposing edges of the fluid permeable body. The method includes removing the metal member from the mandrel. The method includes removing the mandrel from the fluid permeable body.

In an embodiment, a fluid collection device includes a fluid impermeable barrier at least partially defining a chamber. The fluid impermeable barrier includes an opening in fluid communication with the chamber and an aperture in fluid communication with the chamber. The opening is configured to be positioned at least proximate to or receive therein a urethra of a user. The fluid collection device includes a fluid permeable body positioned or positionable within the chamber of the fluid impermeable barrier to extend at least partially across the opening. The fluid permeable body includes an inner layer including a fluid permeable non-woven material, and an outer layer secured to the inner layer with an adhesive and including one or more of a bamboo material, a cotton material, and/or a non-woven hydrophilic material.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection device, according to an embodiment.

FIG. 1B is a front view of a female user wearing the fluid collection device of FIG. 1A.

FIG. 1C is an exploded isometric view of the female fluid collection device of FIG. 1A.

FIG. 1D is a cross-sectional view of the female fluid collection device of FIG. 1A taken along line 1-1 thereof, according to various embodiments.

FIG. 1E is a block diagram of a system for fluid collection, according to an embodiment.

FIG. 2A is a side view of the fluid permeable body of a fluid collection device rolled around a mandrel, according to an embodiment.

FIG. 2B is a side view of a metal member configured to secure to the mandrel of FIG. 2A, according to an embodiment.

FIG. 3 is a flow diagram of a method of manufacturing a fluid permeable body for a fluid collection device, according to an embodiment.

FIG. 4 is a flow diagram of a method of manufacturing a fluid collection device, according to an embodiment.

### DETAILED DESCRIPTION

Fluid collection devices are used to collect fluids such as urine, vaginal discharge, penile discharge, reproductive fluids, blood, sweat, or other bodily fluids from a user. In many embodiments, the fluid collection devices include a fluid permeable member disposed at least partially in a chamber defined by a fluid impermeable barrier. Disclosed herein are methods of manufacturing a fluid collection device and, more specifically, manufacturing a fluid permeable body. In at least one, some, or all embodiments disclosed herein, the methods of manufacturing a fluid permeable body of the fluid collection device result in the technical effect of a dramatically reduced labor and/or material costs during manufacture. In at least one, some, or all embodiments disclosed herein, the fluid permeable body includes an inner layer including a fluid permeable foam material and an outer layer secured to the inner layer. The outer layer may include one or more of a bamboo material, a cotton material, and/or a non-woven hydrophilic material. The resulting fluid permeable body results in the technical effect of a more comfortable fluid collection device in at least one, some, or all embodiments. The outer layer of the fluid permeable body may provide a soft top liner that is able to wick fluids due to the hydrophilic properties of the outer layer, while the inner layer of the fluid permeable foam material may include a hydrophobic material that can easily hold and transfer fluid under vacuum conditions, thereby keeping the fluid collection device in a drying condition while in use and preventing skin irritation and/or skin damage associated with prolonged applications of wet fluid collection devices.

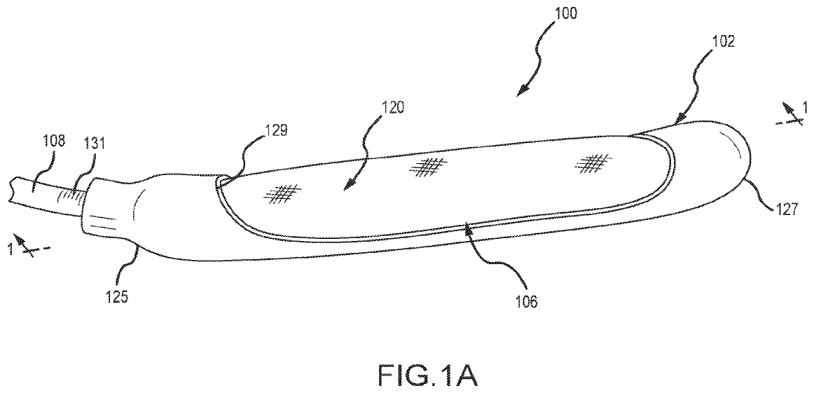

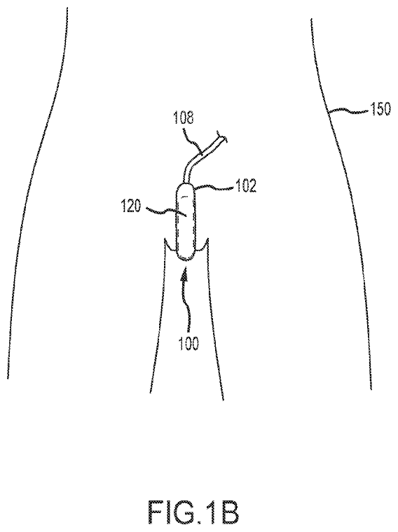

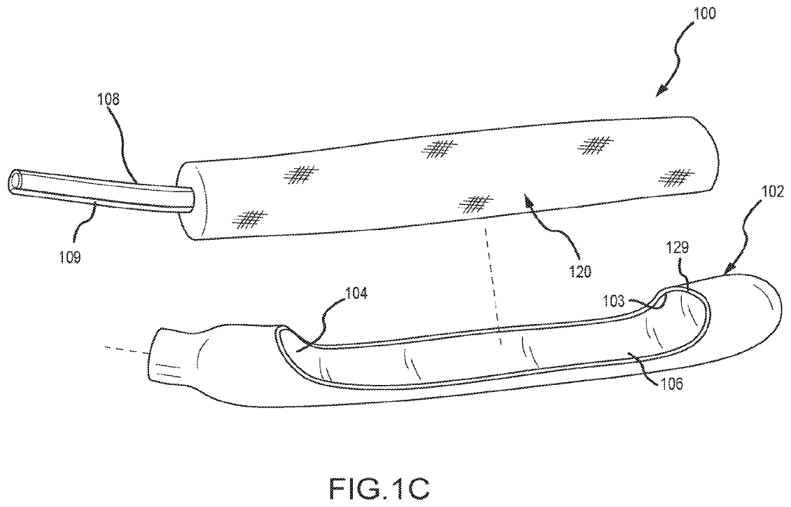

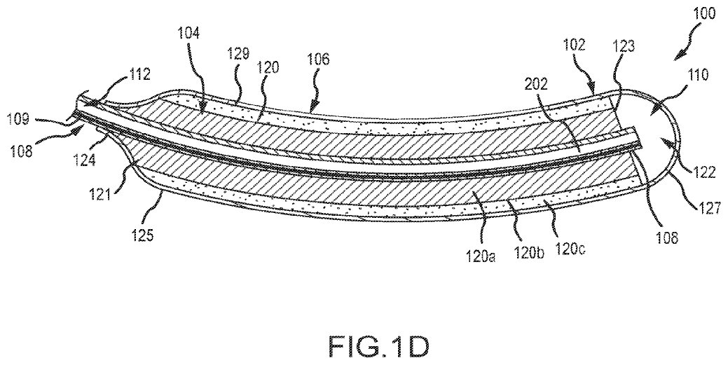

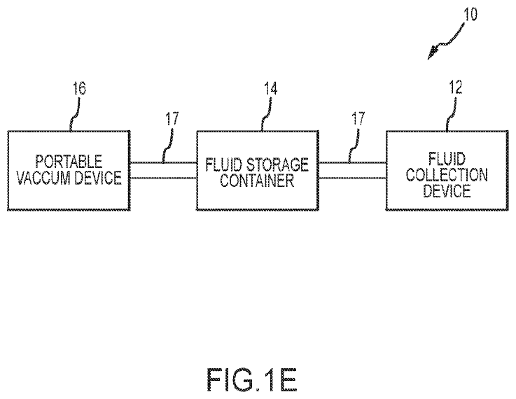

FIGS. 1A-1D show a fluid collection device 100, according to some embodiments. FIG. 1A is an isometric view of a fluid collection device 100, according to an embodiment. The fluid collection device 100 is an example of a female fluid collection device 100 that is configured to receive fluids from a female. The fluid collection device 100 includes a fluid impermeable barrier 102 and a fluid permeable body 120 disposed at least partially within the fluid impermeable barrier 102. FIG. 1B is a front view of a fluid collection device 100 in use on a female user 150. In use, the fluid permeable body 120 of the fluid collection device is positioned at least proximate to a urethra of the user 150. The fluid permeable body 120 is disposed within the chamber 104 (shown in FIG. 1D) of the fluid impermeable barrier 102 of the fluid collection device 100 and is exposed to the urethra of the user 150 through the opening 106 in the fluid collection device 100. The fluid collection device 100 may be secured to the user with any of a number of securement systems disclosed herein. Fluids received in the chamber 104 of the fluid collection device 100 from the urethra may be removed through the conduit 108.

The fluid impermeable barrier 102 may include a first end region 125 and a second end region 127. The fluid impermeable barrier 102 at least partially defines a chamber 104 (e.g., interior region, shown in FIG. 1C) and includes an inward border or edge 129 defining an opening 106. The fluid impermeable barrier 102 is substantially cylindrical in shape between the first end region 125 and the second end region 127. In other embodiments, the fluid impermeable barrier 102 may include other shapes, such as one of more substantially planar surfaces, triangular, or other suitable shape. The opening 106 is formed in and extends longitudinally through the fluid impermeable barrier 102, thereby enabling fluids to enter the chamber 104 from outside of the fluid collection device 100. The opening 106 may be configured to be positioned at least proximate (e.g., adjacent to, interfacing, or contacting) the opening of a female urethra or over a penis of a male. With the fluid collection device 100 may positioned at least proximate to the opening of the female urethra or over a penis, urine may enter the interior region of the fluid collection device 100 via the opening 106. The fluid collection device 100 is configured to receive the fluids into the chamber 104 via the opening 106. For example, the opening 106 may exhibit an elongated shape that is configured to extend from a first location below the urethral opening (e.g., the perineum, at or near the anus, or below the vaginal opening) to a second location above the urethral opening (e.g., at or near the pubic bone). In some embodiments, the opening 106 may exhibit an elongated shape such that about one-third of the opening 106 is above the urethra and about two-thirds of the opening is below the opening 106 during use. The opening 106 may exhibit an elongated shape since the space between the legs of a female is relatively small, thereby only permitting the flow of the fluids along a path that corresponds to the elongated shape of the opening 106. For example, the opening may extend longitudinally along the fluid impermeable barrier. The opening 106 in the fluid impermeable barrier 102 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 since the vacuum (e.g., suction) through the conduit 108 pulls the fluid into the conduit 108. In some embodiments, the opening 106 may be vertically oriented (e.g., having a major axis parallel to the longitudinal axis of the device 100). In some embodiments, (not shown), the opening 106 may be horizontally oriented (e.g., having a major axis perpendicular to the longitudinal axis of the device 100). In some embodiments, the inward border or edge 129 of the fluid impermeable barrier 102 defines the opening 106. The edge 129 may include two opposing arced portions, the arc portions following the outer circumference or periphery of the substantially cylindrical fluid impermeable barrier 102. In an embodiment, the fluid impermeable barrier 102 may be configured to be attached to the individual, such as adhesively attached (e.g., with a hydrogel adhesive) to the individual.

The fluid impermeable barrier 102 may also temporarily store the fluids in the chamber 104. For example, the fluid impermeable barrier 102 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. As such, the fluid impermeable barrier 102 substantially prevents the fluids from exiting the portions of the chamber 104 that are spaced from the opening 106. The fluid impermeable barrier 102 is flexible, thereby enabling the fluid collection device 100 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone.

In an embodiment, the fluid impermeable barrier 102 may be air permeable. In such an embodiment, the fluid impermeable barrier 102 may be formed of a hydrophobic material that defines a plurality of pores. In an embodiment, one or more portions of at least the outer surface of the fluid impermeable barrier 102 may be formed from a soft and/or smooth material, thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

The fluid collection device 100 may include a fluid permeable body 120 or layer disposed in the chamber 104. The fluid permeable body 120 may cover or extend across at least a portion (e.g., all) of the opening 106. The fluid permeable body 120 may be configured to wick or otherwise draw any fluid away from the opening 106, thereby preventing the fluid from escaping the chamber 104. The fluid permeable body 120 also may wick the fluid generally towards an interior of the chamber 104, as discussed in more detail below. A portion of the fluid permeable body 120 may define a portion of an outer surface of the fluid collection device 100. Specifically, the portion of the fluid permeable body 120 defining the portion of the outer surface of the fluid collection device 100 may be the portion of the fluid permeable body 120 exposed by the opening 106 defined by the fluid impermeable barrier 102 that contacts the user. Moreover, the portion of the fluid permeable device defining the portion of the outer surface of the fluid collection device 100 may be free from coverage by gauze or other wicking material at the opening.

The fluid permeable body 120 can be configured to wick and/or allow transport of fluid away from the opening 106 towards a reservoir 122 and/or an inlet 110 of the conduit 108. The fluid permeable body 120 may include any material that may wick the fluid. The permeable properties referred to herein may be wicking, capillary action, diffusion, or other similar properties or processes, and are referred to herein as “permeable” and/or “wicking.” Such “wicking” or other physical properties may exclude absorption into the fluid permeable body 120, such as not include absorption of the bodily fluid into the fluid permeable body 120. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the fluid permeable body 120 (e.g., absorbency), such as less than about 30 wt % of the dry weight of the fluid permeable body 110, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable body 210. In an embodiment, the fluid permeable body 210 may include at least one absorbent or adsorbent material.

The fluid permeable body 120 may include a one-way fluid movement fabric. As such, the fluid permeable body 120 may remove fluid from the area around the female urethra, thereby leaving the urethra dry. FIG. 1D is a cross-sectional view of the fluid collection device 100 taken along line 1-1 of FIG. 1A. The fluid permeable body 120 may enable the fluid to flow generally towards a reservoir 122 (shown in FIG. 1D) in the chamber 104 and/or the inlet 110 of the conduit 108. In some embodiments, the fluid permeable body 120 may include two or more layers of fluid permeable materials. For example, the fluid permeable body 120 may include an inner layer 120a and an outer layer 120c secured to the inner layer 120a.

In some embodiments, inner layer 120a of the fluid permeable body 120 may be a porous layer that includes a non-woven fluid permeable material, such as one or more of polyester, nylon, polypropylene, polyethylene, cellulose, and/or combinations thereof. In some embodiments, the inner layer may include a foam material, such as a non-woven fluid permeable foam material, a synthetic fiber, and/or a hydrophobic material. In some embodiments, the non-woven material of the inner layer 120a may include a vertical non-woven material or normal fiber laid foams. For example, the inner layer 120a may include (as vertical or normal fiber laid foams) a polyester non-woven foam, polypropylene non-woven foam, nylon non-woven foam, polyurethane foam, PVC foam, spacer fabric, and any combinations thereof. A vertical non-woven material, for example, includes a non-woven web that is folded vertically. In an embodiment, the vertical non-woven material may be hydrophobic when the vertical non-woven material exhibits a contact angle with water that is about 90° to about 120°, about 105° to about 135°, about 120° to about 150°, about 135° to about 165°, or greater than 150°.

Generally, increasing the density of the vertical non-woven material increases the strength of the vertical non-woven material. However, increasing the density of the vertical non-woven material may decrease the porosity of the vertical non-woven material which decreases the quantity of bodily fluids that may be temporarily stored in the inner layer 120a and decreases the flow rate of the bodily fluids through the vertical non-woven material. As such, the density of the vertical non-woven material may be selected based on balancing the desired strength, porosity, and flow rate of the bodily fluids through the vertical non-woven material.

In embodiments of the inner layer 120a including the non-woven vertical material, the inner layer may exhibit a surface density of about 100 g/m²/cm to about 250 g/m²/cm, about 100 g/m²/cm to about 175 g/m²/cm, about 175 g/m²/cm to about 250 g/m²/cm, about 100 g/m²/cm to about 150 g/m²/cm, about 150 g/m²/cm to about 200 g/m²/cm, about 200 g/m²/cm to about 250 g/m²/cm, about 100 g/m²/cm to about 125 g/m²/cm, about 125 g/m²/cm to about 150 g/m²/cm, about 150 g/m²/cm to about 175 g/m²/cm, about 175 g/m²/cm, about 175 g/m²/cm to about 200 g/m²/cm, about 200 g/m²/cm to about 225 g/m²/cm, or about 225 g/m²/cm to about 250 g/m²/cm.

The vertical non-woven material of the inner layer 120a may include fibers. The average length, average lateral dimension, and the average aspect ratio of the fibers in the inner layer 120a may be selected based on a number of factors. In an example, increasing the aspect ratio (e.g., increasing average length) of the fibers in the inner layer 120a may increase the mechanical binding of the fibers in the inner layer 120a. For instance, increasing the aspect ratio of the fibers in the inner layer 120a facilitates entanglement of the fibers in the inner layer 120a which increases the strength and durability of the vertical non-woven material. The entanglement of the fibers in the inner layer 120a may also preclude or minimize the amount of other binding techniques that are applied to the vertical non-woven material, such as heat, chemical binding, or other mechanical binding (e.g., further entanglement caused by needle punching or high pressure water jets). However, increasing the aspect ratio of the fibers in the inner layer 120a may make dispersion of the fibers in the inner layer 120a more difficult (e.g., uniformity of the vertical non-woven material difficult). Further, increasing the aspect ratio may limit the type of non-woven webs that may form the vertical non-woven material. For instance, fibers in the inner layer 120a with large average lengths (e.g., large aspect ratios) may not be used in carded webs and may have to be used in air laid webs. In an example, decreasing the aspect ratio may decrease the entanglement of the fibers in the inner layer 120a thereby necessitating further binding of the fibers in the inner layer 120a. As such, the average length, average lateral dimension, and average aspect ratio of the fibers 124 may be selected based on the desired strength, mechanical binding between the fibers in the inner layer 120a, the amount of processing of the vertical non-woven material (e.g., is further processing to increasing the binding via heat, etc. desired), the type of non-woven web that includes the fibers, the uniformity of the fibers, etc.

Generally, the average person discharges urine at a rate of about 6 ml/s to about 50 ml/s, such as at a rate of about 10 ml/s to about 25 ml/s. The rate at which the person urinate may vary, such as based on the size of the person and the age of the person. The vertical non-woven material in the inner layer 120a may be selected to capture and transport the bodily fluids at a rate that is comparable to the rate at which the individual discharged bodily fluids to prevent leaks. For example, the vertical non-woven material may be selected to capture and transport the bodily fluids at a rate that is greater than about 6 ml/s, greater than about 10 ml/s, greater than about 20 ml/s, greater than about 30 ml/s, greater than about 40 ml/s, greater than about 50 ml/s, or in ranges of about 6 ml/s to about 10 ml/s, about 8 ml/s to about 12 ml/s, about 10 ml/s to about 15 ml/s, about 12.5 ml/s to about 17.5 ml/s, about 15 ml/s to about 20 ml/s, about 17.5 ml/s to about 22.5 ml/s, about 20 ml/s to about 25 ml/s, about 22.5 ml/s to about 27.5 ml/s, about 25 ml/s to about 30 ml/s, about 27.5 ml/s to about 35 ml/s, about 30 ml/s to about 40 ml/s, about 35 ml/s to about 45 ml/s, or about 40 ml/s to about 50 ml/s.

The rate at which the vertical non-woven material captures and transports the bodily fluids may depend on a number of factors. In an example, the rate at which the vertical non-woven material captures and transports the bodily fluids may depend inversely on the density and weight basis of the vertical non-woven material, wherein increasing the density and/or weight basis of the vertical non-woven material may decrease the rate at which the vertical non-woven material captures and transports the bodily fluids and vice versa. In an example, the rate at which the vertical non-woven material captures and transports the bodily fluids may depend on the material (e.g., hydrophobicity of the material) that forms the fibers in the inner layer 120a. In an example, the rate at which the vertical non-woven material captures and transports the bodily fluids may increase with increasing thickness T since increasing the thickness T increases the cross-sectional area through which the bodily fluids may flow. In an example, the rate at which the vertical non-woven material captures and transports the bodily fluids may depend on the type of non-woven web (e.g., carded web, needle punched web, etc.) since each type of non-woven web may exhibit different rate at which the vertical non-woven material captures and transports the bodily fluids.

In some embodiments, the vertical non-woven material is formed from a non-woven web that is folded. The folded non-woven web may include a plurality of folded portions and a plurality of intermediate portions extending between the folded portions. The folded non-woven web may include an outer surface adjacent to the fluid impermeable layer and an opposing inner surface (e.g., defining a bore that received the conduit 108). The folded portion may extend generally parallel to the outer and inner surfaces of folded non-woven web. The intermediate portions may extend between the outer and inner surfaces of the folded non-woven web. In an embodiment, the folded non-woven web may be positioned in the chamber such that the folded portions extend generally parallel to a longitudinal (e.g., central) axis of the fluid collection assembly (e.g., generally parallel to a longitudinal axis of the porous material) and/or extend circumferentially when the porous material exhibits a generally cylindrical shape. The folded non-woven web may be positioned in the chamber such that the intermediate portions extend generally parallel to the longitudinal axis of the fluid collection assembly (e.g., generally parallel to a longitudinal axis of the porous material) and/or extend radially when the porous material exhibits a generally cylindrical shape.

As previously discussed, in some embodiments, the inner layer 120a includes vertical non-woven material that includes a plurality of fibers. In an embodiment, the non-woven web that forms the vertical non-woven material may include a plurality of generally oriented fibers. The generally oriented fibers may improve the ability of the vertical non-woven material to capture bodily fluids and transport bodily fluids. The generally oriented fibers may also improve the mechanical properties of the vertical non-woven material. As used herein, the fibers are “generally aligned” when a certain percentage of the fibers are substantially parallel to each other. The certain percentage of the fibers refers to at least about 70% of the fibers, more preferably at least about 80% of the fibers, more preferable 90% of the fibers, and even more preferably at least about 95% of the fibers. The fibers are generally parallel to each other when the certain percentage of fibers are parallel to each other ±30° more preferable ±20°, more preferably ±10°, or even more preferably ±5°.

The non-woven material 118 of the inner layer 120a may be disposed in the chamber 104 such that the fibers of the folded portions are generally oriented circumferentially and the fibers of the intermediate portions are generally oriented radially. Not wishing to be bound to theory, the circumferentially orientation of the fibers of the folded portions may cause the bodily fluids received by the vertical non-woven material to initially preferentially disperse circumferentially and the radial orientation of the fibers of the intermediate portions may cause the bodily fluids to initially preferentially disperse radially into the porous material. Causing the bodily fluids to initially disperse circumferentially and radially quickly disperses the bodily fluids throughout a large volume of the vertical non-woven material thereby allowing the vertical non-woven material to quickly capture and transport the bodily fluids. It is noted that the fibers do not inhibit flow of the bodily fluids in a direction that is generally parallel to the longitudinal axis, especially after the fibers are wetted. Further, dispersing the bodily fluids throughout the vertical non-woven material increases the surface area of any bodily fluids that may remain in the vertical non-woven material after removing the bodily fluids from the porous material. The large surface area facilitates evaporation of the remaining bodily fluids with the air flow through the porous material. In an embodiment, the fibers are randomly oriented or may be oriented differently than described above.

In an embodiment, folding the non-woven web may cause the formation of gaps that extending generally parallel to the longitudinal axis. The gaps may facilitate fluid flow in a direction that is generally parallel to the longitudinal axis. However, the non-woven web may be folded or compressed by the fluid impermeable barrier 102 to minimize the size of the gaps to prevent pooling of the bodily fluids in the chamber 104. For example, the non-woven web may be folded or compressed by the fluid impermeable layer 102 to cause the gaps to exhibit a dimension measured perpendicular to the longitudinal axis that is less than about 1 mm, less than about 0.75 mm, less than about 0.5 mm, or less than about 0.25 mm.

As previously discussed, the vertical non-woven material may be formed from at least one non-woven web that is folded. The vertical non-woven material may be formed from any suitable non-woven web. In an embodiment, the non-woven web includes at least one carded web. The carded web includes a plurality of fibers that may be generally oriented in the same direction. The generally same orientation of the fibers of the carded web cause the carded web to be anisotropic. For example, the strength of the carded web is greatest when a force applied thereto is generally parallel to the fibers but the strength of the carded web decreases as the force applied thereto becomes more oblique or perpendicular to the orientation of the fibers. As such, the carded web may need to be positioned in the chamber 104 to mitigate forces being applied to the carded web that are not generally parallel to the orientation of the fibers or requires addition binding between the fibers (e.g., heat or chemical) to prevent unsatisfactory wear of the carded web. The initial flow the bodily fluids through the carded web may vary depending on whether the bodily fluids are flowing parallel, obliquely, or perpendicular to the orientation of the fibers. As such, selecting the non-woven web to include the carded web allows for selecting the strength and flow characteristics of the porous material based on the orientation of the fibers. Even though the fibers are generally oriented, the orientation of each of the fibers may slightly vary which causes the porosity of the carded web to be sufficiently high that the carded web exhibits any of the density, thickness, basis weight, and flow rates disclosed herein.

In an embodiment, the non-woven web may include at least one needle punched web. The needle punched web may be formed from a sheet including a plurality of fibers. The sheet may include a plurality of randomly oriented fibers (e.g., the fibers are generally parallel to and randomly oriented in the plane), or generally oriented fibers (e.g., a carded web) since the orientation of the fibers may better facilitate flow of the bodily fluids therethrough. A plurality of needles (e.g., a plurality of barbed needles) are inserted into the sheet in a direction that is generally parallel to a thickness of the sheet which causes some of the fibers to become entangled and interlocked. For example, insertion of the needles into the sheet cause some of the fibers to reorient and migrate from the surface of the sheet to an interior thereof to form columns. The entanglement of the fibers caused by the insertion of the needles may sufficiently entangle the fibers such that no additional binding is necessary to bond the fibers together. The entanglement of the fibers may cause the needle punched web to exhibit more isotropic properties compared to the carded web and, thus, may not require specific orientation in the chamber 104 or additional binding of the fibers. The needle punched web may exhibit good flow features. For example, the needles extending into the sheet may form divots which facilitate flow of the bodily fluids vertically through the needle punched web.

In an embodiment, the non-woven web may include at least one air laid web. The air laid web may exhibit a plurality of randomly oriented fibers. The plurality of random fibers may exhibit a length that is sufficiently large that the fibers become entangled and do not need be bounded together or the fibers may be bonded. Due to the random orientation of the fibers, the air laid web tends to be isotropic and exhibit a high porosity. Similar, due to the random orientation of the fibers the air laid web may exhibit a high loft. The air laid web may be formed from fibers that cannot be carded (e.g., short fibers).

In an embodiment, the non-woven web may include at least one spunlaced web. The spunlaced web is formed by providing a sheet that includes randomly oriented fibers or a carded web. High pressure water jets that are generally parallel to the thickness of the sheet are directed towards the sheet. Similar to the needle punched web, the high pressure jets of water cause some of the fibers to migrate from an exterior of the sheet to an interior thereof to form columns. Thus, the spunlaced web may function similar to the needle punched web, namely that the spunlaced web may be more isotropic than the carded web and includes divots. However, the spunlaced web may exhibit at least one of a density that is less than, a thickness that is greater than, or a base weight that is less than the needle punched web. As such, the spunlaced web may be more delicate (e.g., less durable or softer) than the needle punched web. The more delicate spunlaced web may more comfortably contact the skin of the patient than the needle punched web.

In an example, the vertical non-woven material of the inner layer 120a may include a wet laid web even though the wet laid web may exhibit low durability compared to the other non-woven webs disclosed herein. In an example, the vertical non-woven material may include spunbond or meltblown non-woven webs even though such non-woven webs may exhibit too low of porosity for some applications.

In an embodiment, the non-woven web does not include a horizontal or cross lapped non-woven material. The horizontal or cross lapped non-woven material include non-woven webs that are folded differently than the vertical non-woven materials discussed above. It has been found that the different folds of the horizontal and cross lapped non-woven materials causes the horizontal and cross lapped non-woven materials to capture and/or transport bodily fluids significantly less quickly than a comparable (e.g., exhibit same material, hydrophilicity, basis weight, density, etc.) vertical non-woven material. In an embodiment, the non-woven web includes a horizontal or cross lapped non-woven material.

The folded non-woven web may be formed from a sheet. When resting the sheet on a horizontal planar surface, the folded portions may extend parallel to the horizontal planar surface and the intermediate portions may extend vertically from the horizontal planar surface. The folded non-woven web may then be rolled to form the cylindrical folded non-woven web.

In an embodiment, the inner layer 120a only or substantially only includes the vertical non-woven material. In such an embodiment, the vertical non-woven material may define a bore that is configured to receive the conduit 108 and the vertical non-woven material extends from the bore to the fluid impermeable barrier 102. When the inner layer 120a includes only or substantially only the vertical non-woven material, all of the inner layer 120a is able to quickly capture and transport the bodily fluids. However, it is noted that the inner layer 120a may include at least one additional material even though such additional material may decrease at least one of the ability of the inner layer 120a to capture and/or transport the bodily fluids. Other embodiments and aspects of non-woven material and configurations thereof are disclosed in PCT Patent Application No. PCT/US22/42719, filed on Sep. 7, 2022 the disclosure of which is incorporated herein, in its entirety, by this reference.

The inner layer 120a may include a thickness (as measured from the bore to the adhesive 120b) of about 2 mm to about 25 mm, about 2 mm to about 10 mm, about 7 mm to about 15 mm, about 12 mm to about 20 mm, about 17 mm to about 25 mm, about 2 mm to about 7 mm, about 5 mm to about 10 mm, about 7 mm to about 12 mm, about 10 mm to about 15 mm, about 12 mm to about 17 mm, about 15 mm to about 20 mm, about 17 mm to about 22 mm, or about 20 mm to about 25 mm.

The fluid permeable body 120 also includes an outer layer 120c secured to the inner layer 120a. The outer layer 120c may include one or more of a bamboo material, a cotton material, and/or a non-woven hydrophilic material. More specifically, the outer layer 120c may include one or more of the bamboo material, the cotton material, a non-woven polypropylene hydrophilic material, a non-woven polyethylene hydrophilic material, a non-woven polyester hydrophilic material, and/or combinations thereof (such as a bamboo/cotton mixed material).

The outer layer 120c may be formed as a sheet or liner material in many embodiments. For example, the outer layer 120c may include a thickness (as measured from the adhesive 120b to the outer surface of the fluid permeable body 120) of about 0.1 mm to about 0.4 mm, 0.125 mm to about 0.4 mm, about 0.1 mm to about 0.25 mm, about 0.25 mm to about 0.4 mm, about 0.1 mm to about 0.2 mm, about 0.2 mm to about 0.3 mm, about 0.3 mm to about 0.4 mm, about 0.1 mm to about 0.15 mm, about 0.15 mm to about 0.2 mm, about 0.2 mm to about 0.25 mm, about 0.25 mm to about 0.3 mm, about 0.3 mm to about 0.35 mm, or about 0.35 mm to about 0.4 mm.

The outer layer 120c may exhibit a surface density of about 10 gsm to about 100 gsm, about 10 gsm to about 55 gsm, about 55 gsm to about 100 gsm, about 10 gsm to about 20 gsm, about 20 gsm to about 30 gsm, about 30 gsm to about 40 gsm, about 40 gsm to about 50 gsm, about 50 gsm to about 60 gsm, about 60 gsm to about 70 gsm, about 70 gsm to about 80 gsm, about 80 gsm to about 90 gsm, or about 90 gsm to about 100 gsm.

The inner layer 120a may be secured to the outer layer 120c with an adhesive at 120c. The adhesive may include a hotmelt adhesive such as one or more of polyurethane, acrylic, and/or polyester non-woven web adhesive. In some embodiments, the hotmelt adhesive may include particle size ranges of about 1.0 μm to about 10 μm.

In many embodiments, the fluid permeable body 120 is formed to be generally cylindrical, such that the inner layer 120a and the outer layer 120c are concentric with one another. The outer layer 120c may cover or extend across at least a portion (e.g., all) of the opening 106 when the fluid permeable body 120 is disposed in the chamber 104 of the fluid impermeable barrier 102.

The fluid collection device 100 also includes the conduit 108 that is at least partially disposed in the chamber 104. The conduit 108 (e.g., a tube) includes an inlet 110 at a second end region 127 of the fluid impermeable barrier 102 and an outlet 112 at a first end region 125 of the fluid impermeable barrier 102 positioned downstream from the inlet 110. The conduit 108 provides fluid communication between an interior region of the chamber 104 and a fluid storage container (not shown) or a portable vacuum source (not shown). For example, the conduit 108 may directly or indirectly fluidly couple the interior region of the chamber 104 and/or the reservoir 122 with the fluid storage container or the portable vacuum source.

In the illustrated embodiment, the fluid permeable body 120 defines a bore 202 extending through the fluid permeable body 120 from a first body end 121 of the fluid permeable body 120 to a second body end 123 of the fluid permeable body 120 distal to the first body end 120. In other embodiments, the bore 202 extends only partially into the fluid permeable body from the first body end 121 of the fluid permeable body 120.

In the illustrated embodiment, the conduit 108 is at least partially disposed in the chamber 104 and interfaces at least a portion of the bore 202 of the fluid permeable body 120. For example, the conduit 108 may extend into the fluid impermeable barrier 102 from the first end region 125 (e.g., proximate to the outlet 112) and may extend through the bore 202 to the second end region 127 (e.g., opposite the first end region 125) to a point proximate to the reservoir 122 such that the inlet 110 is in fluid communication with the reservoir 122. For example, in the illustrated embodiment, the inlet 110 extends past the second body end 123 and is positioned in the reservoir 122. However, in other embodiments, the inlet 110 may be positioned flush with or behind the second body end 123 of the fluid permeable body 120 that partially defines the reservoir 122. In some embodiments, the second body end 123 extends to the second end region 127 to substantially fill the chamber 104 and cover the inlet 110. The fluid collected in the fluid collection device 100 may be removed from the interior region of the chamber 104 via the conduit 108. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex.

In some embodiments, the conduit 108 includes a shape memory material 109 fixedly secured thereto and/or embedded therein, such as a shape memory polymer or a metal (e.g., shape memory metal), according to an embodiment. The shape memory material 109 may extend longitudinally along the conduit 108. In many embodiments, the shape memory material 109 is configured to bend and/or contour the conduit 108 and the fluid collection device 100 to the unique shape of the body of the user.

Suitable shape memory materials are composed to adopt an intermediate or permanent shape in response to a stimuli. The stimuli may include an external physical force (e.g., bending force), heat, electrical bias, or a magnetic field. While the term “shape memory” is used to describe some of the “shape memory materials” herein, it should be understood that, in some examples, the material modified by the term “shape memory” may not necessarily need to return to a preselected shape upon application of a stimuli, as understood as the classical definition of the “shape memory material.” Rather, at least some of the shape memory materials herein may simply hold a selected shape when bent, set, or cured into a specific shape and/or when cooled in a specific shape, regardless of the stimuli applied thereto after. The shape memory materials may be returned to the original shape or changed to a new shape by application of stimuli. For example, a metal wire bent to a first shape may be utilized as the shape memory material, whereinafter the metal wire may be modified to a second shape via physical force applied thereto or via heating. In an embodiment, the shape memory material may include metal, such as an

elemental metal, an alloy, or shape memory alloy. Suitable shape memory metals may include standard steels, stainless steel, carbon alloy steel, head treated steel, aluminum, silver, copper, iron, nickel, zinc, tin, beryllium, or the like. Suitable shape memory alloys may include stainless steel; galvanized steel; aluminum alloys; nickel-titanium alloys, such as Nitinol, Ni—Ti—Cu, Ni—Ti, Co, or the like; copper-based alloys such as Cu—Zn—Al, Cu—Al—Ni, Cu—Al—Sn, or the like; Co—Cr—Ni—Mo alloys (e.g., Elgiloy®) or the like; or any other alloy having shape memory characteristics. In a particular embodiment, the shape memory material includes 18-20 gauge steel wire. As explained above, the shape memory metals or alloys may merely be metals or alloys that may be shaped to a selected configuration. In some examples, the shape memory metals or alloys may return to a primary shape when an external stimuli is applied thereto. In some examples, the outer surface of the shape memory metal may be coated with a polymer, anodized, passivated, or otherwise treated to prevent corrosion.

Shape memory polymers (“SMPs”) may include polyurethane-based SMPs such as a copolymer (e.g., copolyester, polyurethane, polyetherester, etc.) including blocks of one or more of poly(ε-caprolactone), polyethyleneterephthalate (PET), polyethyleneoxide (PEO), polyethylene glycol (PEG), polystyrene, polymethylmethacrylate (PMMA), polybutylmethacrylate (PBMA), poly (N,N-butadiene), poly (N-methyl-N-oxazoline), polytetrahydrofuran, or poly (butylene terephthalate); thermoplastic polymers such as polyether ether ketone (PEEK), nylon, acetal, polytetrafluoroethylene (PTFE), polysulphone, or the like; polynorbonene; other deformable polymers; or any other shape memory polymer.

The fluid impermeable barrier 102 may store fluids in the reservoir 122 therein. The reservoir 122 may be an unoccupied portion of the chamber 104 and is void of other material. In some embodiments, the reservoir 122 is defined at least partially by the fluid permeable body 120 and the fluid impermeable barrier 102. For example, in an embodiment, the reservoir 122 may be located at the portion of the chamber 104 that is closest to the inlet 110 (e.g., the second end region). Accordingly, in the embodiment in FIG. 1D, the reservoir 122 is defined by the second body end 123 of the fluid permeable body 120 and the second end region 127 of the fluid impermeable barrier 122. However, the reservoir 122 may be located at different locations in the chamber 104. For example, the reservoir 122 may be located at the end of the chamber 104 that is closest to the outlet 112. In these and other embodiments, the conduit 108 may extend through the first end region 125 of the fluid impermeable barrier 102 and to the reservoir 122 without extending through the fluid permeable body 120. Accordingly, in these and other embodiments, the fluid permeable body 120 may be free from the bore. In another embodiment, the fluid collection device 100 may include multiple reservoirs, such as a first reservoir that is located at the portion of the chamber of the chamber 104 that is closest to the inlet 110 (e.g., second end region) and a second reservoir that is located at the portion of the of the chamber 104 that is closest to the outlet 112 (e.g., first end region). In another example, the fluid permeable body 120 is spaced from at least a portion of the conduit 108 and the reservoir 122 may be the space between the fluid permeable body 120 and the conduit 108. In some embodiments, the fluid permeable body 120 fills or occupies substantially all of the chamber 104, including filling or occupying substantially all of the reservoir 122 is between the inlet 110 and the second end region 127 of the fluid impermeable barrier 102. Other embodiments of reservoirs, fluid impermeable barriers, fluid permeable membranes, fluid permeable bodies, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. patent application Ser. No. 15/611,587 filed on Jun. 1, 2017, the disclosure of each of which is incorporated herein, in its entirety, by this reference.

The fluid impermeable barrier 102 and the fluid permeable body 120 may be configured to have the conduit 108 at least partially disposed in the chamber 104. For example, the fluid permeable body 120 may be configured to form a space that accommodates the conduit 108, such as the bore 202. In another example, the fluid impermeable barrier 102 may define an aperture 124 sized to receive the conduit 108 (e.g., at least one tube). The at least one conduit 108 may be disposed in the chamber 104 via the aperture 124. The aperture 124 may be configured to form an at least substantially fluid tight seal against the conduit 108 or the at least one tube thereby substantially preventing the fluids from escaping the chamber 104.

In some embodiments, the conduit 108 may extend through the fluid permeable body 120 and at least partially into the reservoir 122, as shown in FIG. 1D. In some embodiments, the conduit 108 may extend through the fluid permeable body 120 and terminate at or before the second body end 123 of the fluid permeable body 120 such that the conduit 108 does not extend into the reservoir 122 (or the reservoir 122 is absent of the conduit 108). For example, an end (e.g., the inlet 110) of the conduit 108 may be generally flush or coplanar with the second body end 123 of the fluid permeable body 120. In other embodiments, the end of the conduit 108 may be recessed from the second body end 123 of the fluid permeable body 120. The end (e.g., the inlet 110) of the conduit 108 also may be selectively moveable between partially extending into the reservoir 122 (shown in FIG. 1D) and recessed from or flush with the second body end 123 of the fluid permeable body.

When secured to the fluid collection device 100, the conduit 108 is configured to provide fluid communication with and at least partially extend between one or more of a fluid storage containers and a portable vacuum source. For example, the conduit 108 may be configured to be fluidly coupled to and at least partially extend between one or more of the fluid storage containers and the portable vacuum source. In an embodiment, the conduit 108 is configured to be directly connected to the portable vacuum source. In such an example, the conduit 108 may extend from the fluid impermeable barrier 102 by at least one foot, at least two feet, at least three feet, or at least six feet. In another example, the conduit 108 is configured to be indirectly connected to at least one of the fluid storage container or the portable vacuum source. In some examples, the conduit may be frosted or opaque (e.g., black) to obscure visibility of the fluids therein. In some embodiments, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 110 and the outlet 112 are configured to provide fluid communication (e.g., directly or indirectly) between the portable vacuum source (not shown) and the chamber 104 (e.g., the reservoir 122). For example, the inlet 110 and the outlet 112 of the conduit 108 may be configured to directly or indirectly fluidly couple the portable vacuum source to the reservoir 122. In an embodiment, the inlet 110 and/or the outlet 112 may form a male connector. In another example, the inlet 110 and/or the outlet 112 may form a female connector. In an embodiment, the inlet 110 and/or the outlet 112 may include ribs that are configured to facilitate secure couplings. In an embodiment, the inlet 110 and/or the outlet 112 may form a tapered shape. In an embodiment, the inlet 110 and/or the outlet 112 may include a rigid or flexible material.

Locating the inlet 110 at or near a gravimetrically low point of the chamber 104 enables the conduit to receive more of the fluids than if inlet 110 was located elsewhere and reduce the likelihood of pooling (e.g., pooling of the fluids may cause microbe growth and foul odors). For instance, the fluids in the fluid permeable body 120 may flow in any direction due to capillary forces. However, the fluids may exhibit a preference to flow in the direction of gravity, especially when at least a portion of the fluid permeable body 120 is saturated with the fluids.

As the portable vacuum source applies a vacuum/suction in the conduit 108, the fluid(s) in the chamber 104 (e.g., such as in the reservoir 122 positioned at the first end region 125, the second end region 127, or other intermediary positions within the chamber 104) may be drawn into the inlet 110 and out of the fluid collection device 100 via the conduit 108.

In an embodiment, the conduit 108 is configured to be at least insertable into the chamber 104. In such an embodiment, the conduit 108 may include one or more markers 131 (shown in FIG. 1A) on an exterior thereof that are configured to facilitate insertion of the conduit 108 into the chamber 104. For example, the conduit 108 may include one or more markings thereon that are configured to prevent over or under insertion of the conduit 108, such as when the conduit 108 defines an inlet 110 that is configured to be disposed in or adjacent to the reservoir 122. In another embodiment, the conduit 108 may include one or more markings thereon that are configured to facilitate correct rotation of the conduit 108 relative to the chamber 104. In an embodiment, the one or more markings may include a line, a dot, a sticker, or any other suitable marking. In examples, the conduit 108 may extend into the fluid impermeable barrier 102 from the first end region (e.g., proximate to the outlet 112) and may extend to the second end region (e.g., opposite the first end region) to a point proximate to the reservoir 122 such that the inlet 110 is in fluid communication with the reservoir 122. In some embodiments (not shown), the conduit 108 may enter the second end region and the inlet 110 may be disposed in the second end region (e.g., in the reservoir 122). The fluid collected in the fluid collection device 100 may be removed from the interior region of the chamber 104 via the conduit 108. The conduit 108 may include a flexible material such as plastic tubing (e.g., medical tubing) as disclosed herein. In some examples, the conduit 108 may include one or more portions that are resilient, such as having one or more of a diameter or wall thickness that allows the conduit to be flexible.

In an embodiment, one or more components of the fluid collection device 100 may include an antimicrobial material, such as an antibacterial material where the fluid collection device may contact the wearer or the bodily fluid of the wearer. The antimicrobial material may include an antimicrobial coating, such as a nitrofurazone or silver coating. The antimicrobial material may inhibit microbial growth, such as microbial growth due to pooling or stagnation of the fluids. In an embodiment, one or more components of the fluid collection device 100 (e.g., impermeable barrier 102, conduit 108, etc.) may include an odor blocking or absorbing material such as a cyclodextrine containing material or a thermoplastic elastomer (TPE) polymer.

In any of the embodiments disclosed herein, the conduits 108 may include or be operably coupled to a flow meter (not shown) to measure the flow of fluids therein, one or more securement devices (e.g., a StatLock securement device, not shown) or fittings to secure the conduit 108 to one or more components of the systems or devices disclosed herein (e.g., portable vacuum source or fluid storage container), or one or more valves to control the flow of fluids in the systems and devices herein. In an embodiment, at least one of portion of the conduit 108 of the fluid collection devices or systems herein may be formed of an at least partially opaque material which may obscure the fluids that are present therein. For example, a first section of the conduit 108 disclosed herein may be formed of an opaque material or translucent material while a second section of the conduit 108 may be formed of a transparent material or translucent material. In some embodiments, the first section may include transparent or translucent material. Unlike the opaque or nearly opaque material, the translucent material allows a user of the devices and systems herein to visually identify fluids or issues that are inhibiting the flow of fluids within the conduit 108.

In any of the examples, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device.

FIG. 1E is a block diagram of a system 10 for fluid collection, according to an embodiment. The system 10 includes a fluid collection device 12, a fluid storage container 14, and a portable vacuum source 16. The fluid collection device 12 may include any of the fluid collection devices disclosed herein, such as the fluid collection device 100. The fluid collection device 12, the fluid storage container 14, and the portable vacuum source 16 may be fluidly coupled to each other via one or more conduits 17. The conduit 17 may include any of the conduits disclosed herein, such as the conduit 108. The fluid collection device 12 may be operably coupled to one or more of the fluid storage container 14 or the portable vacuum source via the conduit 17. Fluid (e.g., urine or other bodily fluids) collected in the fluid collection device 12 may be removed from the fluid collection device 12 via the conduit 17, which protrudes into an interior region of the fluid collection device 12. For example, a first open end of the conduit 17 may extend into the fluid collection device 12 to a reservoir therein. The second open end of the conduit 17 may extend into the fluid storage container 14 or the portable vacuum source 16. The suction force may be introduced into the interior region of the fluid collection device 12 via the first open end of the conduit 17 responsive to a suction (e.g., vacuum) force applied at the second end of the conduit 17. The suction force may be applied to the second open end of the conduit 17 by the portable vacuum source 16 either directly or indirectly.

The suction force may be applied indirectly via the fluid storage container 14. For example, the second open end of the conduit 17 may be disposed within the fluid storage container 14 and an additional conduit 17 may extend from the fluid storage container 14 to the portable vacuum source 16. Accordingly, the portable vacuum source 16 may apply suction to the fluid collection device 12 via the fluid storage container 14. The suction force may be applied directly via the fluid storage container 14. For example, the second open end of the conduit 17 may be disposed within the portable vacuum source 16. An additional conduit 17 may extend from the portable vacuum source 16 to a point outside of the fluid collection device 12, such as to the fluid storage container 14. In such examples, the portable vacuum source 16 may be disposed between the fluid collection device 12 and the fluid storage container 14.

The fluid collection device 12 may be shaped and sized to be positioned adjacent or proximate to a female urethra. The fluid collection member of the fluid collection device 12 may include a fluid impermeable barrier at least partially defining a chamber (e.g., interior region of the fluid collection device member) of the fluid collection device 12. As described in more detail above, the fluid collection device 12 may include a softer, thinner fluid impermeable barrier than conventional fluid collection devices. The fluid impermeable barrier also defines an opening extending therethrough from the external environment. The opening may be positioned on the fluid collection member to be aligned adjacent or proximate to a female urethra. The fluid collection member of the fluid collection device 12 may include a fluid permeable body disposed within the fluid impermeable barrier. The fluid permeably body may include a fluid permeable membrane and fluid permeable support disposed within the fluid permeable membrane. The conduit 17 may extend into the fluid collection device 12 at a first end region, through one or more of the fluid impermeable barrier, fluid permeable membrane, or the fluid permeable support to a second end region of the fluid collection member of the fluid collection device 12. Example fluid collection devices for use with the systems and methods herein are described in more detail below.

In some embodiments, the fluid storage container 14 may include a bag (e.g., drainage bag), a bottle or cup (e.g., collection jar), or any other enclosed container for storing bodily fluids such as urine. In examples, the conduit 17 may extend from the fluid collection device 12 and attach to the fluid storage container 14 at a first point therein. An additional conduit 17 may attach to the fluid storage container 14 at a second point thereon and may extend and attach to the portable vacuum source 16. For example, the fluid storage container 14 may include a container fluidly coupled to a first conduit section that is also fluidly coupled to the fluid collection member of the fluid collection device 12. The container may be fluidly coupled to a second section of the conduit 17 that is also fluidly coupled to a portable vacuum source. In such examples, the portable vacuum source 16 may provide a vacuum/suction through the container to the fluid collection member to provide suction in the chamber of the fluid collection member. Accordingly, a vacuum (e.g., suction) may be drawn through fluid collection device 12 via the fluid storage container 14. As the fluid is drained from the chamber, the fluid may travel through the first section of conduit to the fluid storage container where it may be retained. Fluid, such as urine, may be drained from the fluid collection device 12 using the portable vacuum source 16.

In some embodiments, the portable vacuum source 16 may be disposed in or on the fluid collection device 12. In such examples, the conduit 17 may extend from the fluid collection device and attach to the portable vacuum source 16 at a first point therein. An additional conduit 17 may attach to the portable vacuum source 16 at a second point thereon and may extend out of the fluid collection device 12, and may attach to the fluid storage container 14. Accordingly, a vacuum (e.g., suction) may be drawn through fluid collection device 12 via the fluid storage container 14.

The portable vacuum source 16 may include one or more of a manual vacuum pump, and electric vacuum pump, a diaphragm pump, a centrifugal pump, a displacement pump, a magnetically driven pump, a peristaltic pump, or any pump configured to produce a vacuum. The portable vacuum source 16 may provide a vacuum or suction to remove fluid from the fluid collection member of the fluid collection device 12. In some embodiments, the portable vacuum source 16 may be powered by one or more of a power cord (e.g., connected to a power socket), one or more batteries, or even manual power (e.g., a hand operated vacuum pump). In examples, the portable vacuum source 16 may be sized and shaped to fit outside of, on, or within the fluid collection device 12. For example, the portable vacuum source 16 may include one or more miniaturized pumps or one or more micro pumps. The portable vacuum sources 16 disclosed herein may include one or more of a switch, a button, a plug, a remote, or any other device suitable to activate the portable vacuum source 16. It should be understood that the portable vacuum sources 16 disclosed herein may provide a portable means of providing a suction or vacuum that allows use of the devices and systems herein outside of hospital or care facility environments where vacuum lines are plumbed into patient rooms or large (e.g., larger or heavier than a patient can readily carry) vacuum sources are located. For example, a portable vacuum source may be small and light enough to be carried by a user (e.g., patient) or aid (e.g., nurse) during transportation of the user.

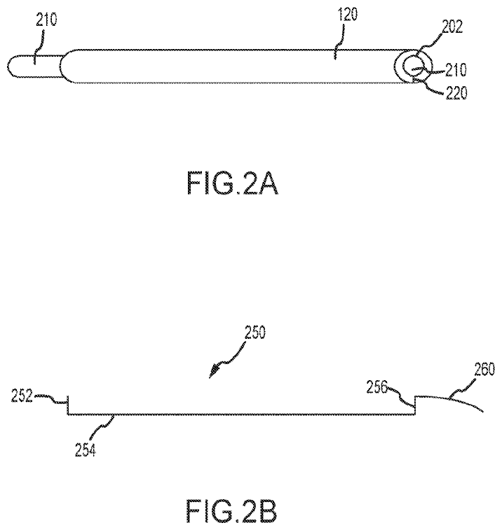

Turning ahead in the drawings, FIG. 2A is a side view of the fluid permeable body 120 of the fluid collection device 100 rolled around a mandrel 210, and FIG. 2B is a side view of a metal member 250 configured to secure to the mandrel 210 of FIG. 2A, according to an embodiment. The mandrel 210 and the metal member 250 may be used in the manufacture of the fluid collection device 100 and/or the fluid permeable body 120, as shall be described in greater detail below. The mandrel 210 may include a rod or cylinder having any of a variety of mandrel configurations and materials. In some embodiments, the mandrel 210 includes an outer diameter of about 0.5 cm to about 2 cm, about 0.5 cm to about 1.5 cm, about 0.5 cm to about 1 cm, about 0.75 cm to about 1.25 cm, about 1 cm to about 1.5 cm, about 0.5 cm, about 0.75 cm, about 1 cm, about 1.25 cm, or about 1.5 cm.

The metal member 250 may include a body of metal string(s) or metal wire and an electrical line 260 electrically coupled to the metal member 250. In some embodiments, the metal member 250 includes a proximal end region 252, an intermediate region 254, and a distal end region 256. The proximal end region 252 and/or the distal end region 256 may be angled (e.g., perpendicular) relative to the intermediate region 254. The proximal end region 252 and the distal end region 256 may be spaced from one another at a length that is substantially equal to a longitudinal length of the fluid permeable body 120 when wrapped around the mandrel 210. The metal member 250 may configured to detachably secure to the mandrel 210. For example, one or more (e.g., both) of the proximal end region 252 and/or the distal end region 256 may be configured to secure to the mandrel 210. When secured to the mandrel, at least one (e.g., all) of the proximal end region 252, the distal end region 256, and/or the intermediate region 254 may contact opposing edges or ends of the fluid permeable body 210 wrapped around the mandrel 210. The metal member 250 may be configured such that, when activated, at least one (e.g., all) of the proximal end region 252, the distal end region 256, and/or the intermediate region 254 may thermally seal or secure opposing edges or ends of the fluid permeable body 210 together. Accordingly, the metal member 250 may be configured to heat to a temperature sufficient to thermally seal edges of at least the outer layer 120c of the fluid permeable body 120.

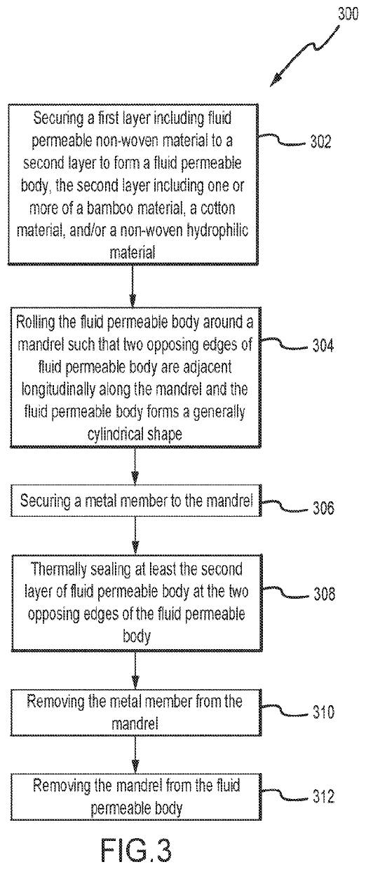

FIG. 3 is a flow diagram of a method 300 of manufacturing a fluid permeable body for a fluid collection device, according to an embodiment. The method 300 may form any embodiment of the fluid permeable body 120 described above. In an embodiment, the method 300 includes an act 302 of securing a first layer to a second layer to form a fluid permeable body. In some embodiments, the method 300 may include an act 304 of rolling the fluid permeable body around a mandrel such that two opposing edges of fluid permeable body are adjacent longitudinally along the mandrel and the fluid permeable body forms a generally cylindrical shape with the first layer being an inner layer and the second layer being an outer layer. In some embodiments, the method 300 also may include an act 306 of securing a metal member to the mandrel with a distal end region of the metal member securing to the mandrel proximate to a distal end of the fluid permeable body, a proximal end region of the metal member proximate to a proximal end of the fluid permeable body, and an intermediate region of the metal member proximate to the two opposing edges of the fluid permeable body. In some embodiments, the method 300 also may include an act 308 of thermally sealing at least the second layer of fluid permeable body at the two opposing edges of the fluid permeable body. In some embodiments, the method 300 may include an act 310 of removing the metal member from the mandrel and an act 312 of removing the mandrel from the fluid permeable body. Acts of the method 300 are for illustrative purposes. For example, the acts of the method 300 may be performed in different orders, split into multiple acts, modified, supplemented, or combined.

In the method 300, the first layer may include any material or configuration of the inner layer 120a described above, and the second layer may include any material or configuration of the outer layer 120b described above. For example, the first layer may include a fluid permeable (e.g., porous) non-woven material and the second layer may include one or more of a bamboo material, a cotton material, and/or a non-woven hydrophilic material. In some embodiments, the second layer may include one or more of the bamboo material, the cotton material, a non-woven polypropylene hydrophilic material, a non-woven polyethylene hydrophilic material, and/or a non-woven polyester hydrophilic material. In some embodiments, the second layer includes a surface density of about 10 gsm to about 100 gsm. In some embodiments, the fluid permeable non-woven material of the first layer includes a vertical non-woven foam material, such as a vertical non-woven foam material that includes one or more of polyester, nylon, polypropylene, polyester, and/or cellulose. The first layer may include a surface density of about 100 g/m²/cm to about 250 g/m²/cm. The second layer may include a thickness of about 0.125 mm to about 0.4 mm and the first layer includes a thickness of about 2 mm to about 25 mm.

In some embodiments, the act 302 of securing a first layer to a second layer to form a fluid permeable body includes hotmelting the first layer to the second layer within a hotmelt adhesive including one or more of polyurethane, acrylic, and/or polyester non-woven web adhesive. For example, in an assembly process, the first layer may be disposed on a transfer line of a belt conveyor and have a hotmelt adhesive web applied to the first layer. The hotmelt adhesive may one or more of thermoplastic, polyurethane, acrylic, and/or polyester non-woven web adhesive. In some embodiments, the hotmelt adhesive may include particles sprayed on top of the first layer on the transfer line. Accordingly, in some embodiments, the method 300 may include spraying the hotmelt adhesive particles on the first layer. Particle sizes in the hotmelt adhesive may include about 1.0 μm to about 10.0 μm, about 1.0 μm to about 5.0 μm, about 5.0 μm to about 10.0 μm, about 1.0 μm to about 3.0 μm, about 3.0 μm to about 5.0 μm, about 5.0 μm to about 7.0 μm, or about 7.0 μm to about 9.0 μm.

The second layer may be applied to the transfer line of the belt conveyor on top of the hotmelt adhesive web such that the hotmelt adhesive web is disposed between the first layer and the second layer. With the hotmelt adhesive web disposed between the first layer and the second layer on the transfer line, the convey belt may be rolled or passed through a hot roller on the transfer line that melts the hotmelt adhesive web to secure the first layer to the second layer.

The hot roller may apply a predetermined temperature to the assembly of the first layer, the hotmelt adhesive, and the second layer. In some embodiments, the hot roller applies a temperature of about 60° C. to about 140° C., about 60° C. to about 100° C., about 100° C. to about 140° C., about 60° C. to about 80° C., about 80° C. to about 100° C., about 100° C. to about 120° C., or about 100° C. to about 140° C.

As provided above, the method 300 may include the act 304 of rolling the fluid permeable body around a mandrel such that two opposing edges of fluid permeable body are adjacent longitudinally along the mandrel and the fluid permeable body forms a generally cylindrical shape with the first layer being an inner layer and the second layer being an outer layer. FIG. 2A shows an example of the fluid permeable body 120 rolled around the mandrel 210 such that two opposing edges 220 of the fluid permeable body are adjacent longitudinally along the mandrel 210 and the fluid permeable body 120 forms a generally cylindrical shape. A bore 202 is formed through the generally cylindrical fluid permeable body 210.

As provided above, in some embodiments, the method 300 also may include an act 306 of securing a metal member to the mandrel with a distal end region of the metal member securing to the mandrel proximate to a distal end of the fluid permeable body, a proximal end region of the metal member proximate to a proximal end of the fluid permeable body, and/or an intermediate region of the metal member proximate to the two opposing edges of the fluid permeable body. FIG. 2B, for example shows the metal member 250 including a proximal end region 252, the intermediate region 254, and the distal end region 256.

In the act 306, the metal member 250 may be secured to the mandrel 210 and/or the fluid permeable body 120 disposed around the mandrel 210. For example, at least one (e.g., both) of the proximal end region 252 and/or the distal end region 256 of the metal member 250 may be secured to the mandrel 210 and/or the fluid permeable body 120. The proximal end region 252 and/or the distal end region 256 may be mechanically secured to the mandrel 210. The distal end region 256 of the metal member 250 may be secured to the mandrel 210 proximate or adjacent to the two opposing edges 220 at the distal end of the fluid permeable body 120, the proximal end region 252 of the metal member 250 may be secured to the mandrel 210 proximate or adjacent to the two opposing edges 220 at the proximal end of the fluid permeable body 120, and/or the intermediate region 254 of the metal member 250 may be disposed proximate to the two opposing edges of the fluid permeable body 120 between the proximal end and the distal end of the fluid permeable body 120.

In some embodiments, the metal member 250 clamps over the fluid permeable body 120 such that the distal end region 256 of the metal member 250 is proximate or adjacent to the two opposing edges 220 at the distal end of the fluid permeable body 120, the proximal end region 252 of the metal member 250 is proximate or adjacent to the two opposing edges 220 at the proximal end of the fluid permeable body 120, and/or the intermediate region 254 of the metal member 250 is proximate to the two opposing edges of the fluid permeable body 120 between the proximal end and the distal end of the fluid permeable body 120.

In some embodiments, the method 300 also may include an act 308 of thermally sealing at least the second layer of fluid permeable body at the two opposing edges of the fluid permeable body. For example, the metal member 250 may be activated by the electrical line 260 to heat to a predetermined temperature. When heated, the distal end region 256 of the metal member 250 may thermally seal at least some of the two opposing edges 220 at the distal end of the fluid permeable body 120, the proximal end region 252 of the metal member 250 may thermally seal at least some of the two opposing edges 220 at the proximal end of the fluid permeable body 120, and/or the intermediate region 254 of the metal member 250 may thermally seal at least some of the two opposing edges of the fluid permeable body 120 between the proximal end and the distal end of the fluid permeable body 120. In some embodiments, the metal member 250 may thermally seal opposing edges of the second layer (e.g., outer layer) together when the metal member 250 is activated.

In some embodiments, the acts 306 and 308 are absent from the method 300. Instead, the method 300 may include an act of securing together the two opposing edges of the fluid permeable body with an adhesive or double-sided tape.

Acts of the method 300 are for illustrative purposes. For example, the acts of the method 300 may be performed in different orders, split into multiple acts, modified, supplemented, or combined.

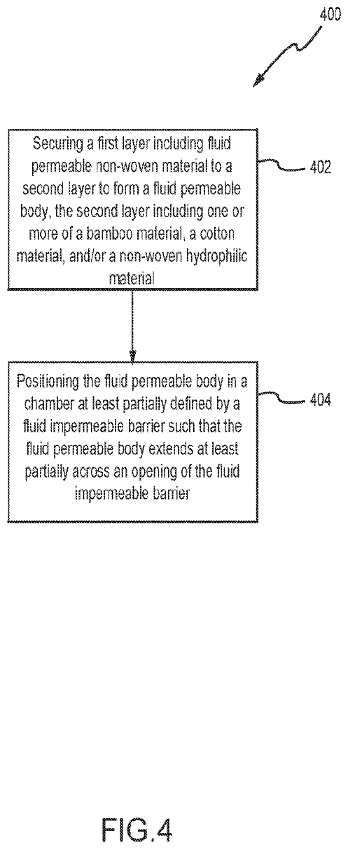

FIG. 4 is a flow diagram of a method 400 of manufacturing a fluid collection device, according to an embodiment. The method 400 may form any embodiment of the fluid permeable body 120 and/or the fluid collection device 100 described above. The method 400 may include an act 402 of securing a first layer to a second layer to form a fluid permeable body. The method 400 also may include an act 404 of positioning the fluid permeable body in a chamber at least partially defined by a fluid impermeable barrier such that the fluid permeable body extends at least partially across an opening of the fluid impermeable barrier.

The act 402 of securing a first layer to a second layer to form a fluid permeable body may include any aspect of the act 302 of securing a first layer to a second layer to form a fluid permeable body described above in relation to the method 300. In the method 400, the first layer may include any aspect or material of the first layer in the method 300 or the inner layer 120a of the fluid collection device 100. In the method 400, the second layer may include any aspect or material of the second layer in the method 300 or the outer layer 120c of the fluid collection device 100. Furthermore, the method 400 may include any aspect of the method 300 described above in forming a fluid permeable body. For example, the method 400 may include one or more (e.g., all) of acts 304, 306, 308, 310, and/or 312 of the method 300.

In some embodiments, the method 400 further includes inserting a conduit through a bore in the fluid permeable body. The conduit may include a shape memory material fixedly secured thereto and/or embedded therein, such as the shape memory material 109. The method 400 may further include inserting the conduit through the aperture in the fluid impermeable barrier.

Acts of the method 400 are for illustrative purposes. For example, the acts of the method 400 may be performed in different orders, split into multiple acts, modified, supplemented, or combined.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

What is claimed is:

1. A method of manufacturing a fluid collection device, the method comprising:
securing a first layer including fluid permeable non-woven material to a second layer with an adhesive to form a fluid permeable body, the second layer including one or more of a bamboo material, a cotton material, and/or a non-woven hydrophilic material; and
positioning the fluid permeable body in a chamber at least partially defined by a fluid impermeable barrier such that the fluid permeable body extends at least partially across an opening of the fluid impermeable barrier.

2. The method of claim 1, wherein the second layer includes one or more of the bamboo material, the cotton material, a non-woven polypropylene hydrophilic material, a non-woven polyethylene hydrophilic material, and/or a non-woven polyester hydrophilic material.

3. The method of any of claim 1 or 2, wherein the second layer includes a surface density of about 10 gsm to about 100 gsm.

4. The method of any of claims 1-3, wherein the fluid permeable non-woven material of the first layer includes a vertical non-woven foam material.

5. The method of claim 4, wherein the vertical non-woven foam material includes one or more of polyester, nylon, polypropylene, polyethylene, and/or cellulose.

6. The method of any of claims 1-5, wherein the first layer includes a surface density of about 100 g/m²/cm to about 250 g/m²/cm.

7. The method of any of claims 1-6, wherein the second layer includes a thickness of about 0.125 mm to about 0.4 mm and the first layer includes a thickness of about 2 mm to about 25 mm.

8. The method of any of claims 1-7, wherein securing a first layer including fluid permeable non-woven material to a second layer with an adhesive to form a fluid permeable body includes hotmelting the first layer to the second layer with the hotmelt adhesive including one or more of thermoplastic, polyurethane, acrylic, and/or polyester non-woven web adhesive.

9. The method of claim 8, further comprising spraying the hotmelt adhesive on the first layer before hotmelting the first layer to the second layer with the hotmelt adhesive positioned between the first layer and the second layer.

10. The method of any of claims 1-9, further comprising rolling the fluid permeable body around an axis and securing two edges of the fluid permeable body together such that the fluid permeable body is substantially cylindrical with the first layer being an inner layer and the second layer being an outer layer concentric with the inner layer, wherein the fluid impermeable barrier is substantially cylindrical.

11. The method of any of claims 1-9, further comprising:
rolling the fluid permeable body around a mandrel such that two opposing edges of fluid permeable body are adjacent longitudinally along the mandrel and the fluid permeable body forms a generally cylindrical shape;
securing a metal member to the mandrel with a distal end of the metal member securing to the mandrel proximate to a distal end of the fluid permeable body, a proximal end of the metal member proximate to a proximal end of the fluid permeable body, and an intermediate region of the metal member proximate to the two opposing edges of the fluid permeable body;
thermally sealing at least the second layer of fluid permeable body at the two opposing edges of the fluid permeable body;
removing the metal member from the mandrel; and
removing the mandrel from the fluid permeable body.

12. The method of any of claims 1-11, further comprising:
inserting a conduit through a bore in the fluid permeable body, the conduit including a shape memory material fixedly secured thereto and/or embedded therein; and
inserting the conduit through the aperture in the fluid impermeable barrier.

13. A method of manufacturing a fluid permeable member for a fluid collection device, the method comprising:
securing a first layer including fluid permeable non-woven material to a second layer to form a fluid permeable body, the second layer including one or more of a bamboo material, a cotton material, and/or a non-woven hydrophilic material;
rolling the fluid permeable body around a mandrel such that two opposing edges of fluid permeable body are adjacent longitudinally along the mandrel and the fluid permeable body forms a generally cylindrical shape;
securing a metal member to the mandrel with a distal end of the metal member securing to the mandrel proximate to a distal end of the fluid permeable body, a proximal end of the metal member proximate to a proximal end of the fluid permeable body, and an intermediate region of the metal member proximate to the two opposing edges of the fluid permeable body;
thermally sealing at least the second layer of fluid permeable body at the two opposing edges of the fluid permeable body;
removing the metal member from the mandrel; and
removing the mandrel from the fluid permeable body.

14. The method of claim 12, wherein the second layer includes one or more of the bamboo material, the cotton material, a non-woven polypropylene hydrophilic material, a non-woven polyethylene hydrophilic material, and/or a non-woven polyester hydrophilic material.

15. The method of any of claim 12 or 13, wherein the second layer includes a surface density of about 10 gsm to about 100 gsm.

16. The method of any of claims 12-14, wherein the fluid permeable non-woven material of the first layer includes a vertical non-woven foam material.

17. The method of claim 15, wherein the vertical non-woven foam material includes one or more of polyester, nylon, polypropylene, polyester, and/or cellulose.

18. The method of any of claims 12-16, wherein the first layer includes a surface density of about 100 g/m²/cm to about 250 g/m²/cm.

19. The method of any of claims 12-17, wherein the second layer includes a thickness of about 0.125 mm to about 0.4 mm and the first layer includes a thickness of about 2 mm to about 25 mm.

20. The method of any of claims 12-18, wherein securing a first layer including fluid permeable non-woven material to a second layer to form a fluid permeable body includes hotmelting the first layer to the second layer within a hotmelt adhesive including one or more of polyurethane, acrylic, and/or polyester non-woven web adhesive.

21. The method of claim 20, further comprising spraying the hotmelt adhesive on the first layer before hotmelting the first layer to the second layer with the hotmelt adhesive positioned between the first layer and the second layer.

22. A fluid collection device, comprising:
a fluid impermeable barrier at least partially defining a chamber, the fluid impermeable barrier including an opening in fluid communication with the chamber and an aperture in fluid communication with the chamber, the opening being configured to be positioned at least proximate to or receive therein a urethra of a user; and
a fluid permeable body positioned within the chamber of the fluid impermeable barrier to extend at least partially across the opening, the fluid permeable body including an inner layer including a fluid permeable non-woven material, and an outer layer secured to the inner layer with an adhesive and including one or more of a bamboo material, a cotton material, and/or a non-woven hydrophilic material.

23. The fluid collection device of claim 20, wherein the outer layer includes one or more of the bamboo material, the cotton material, a non-woven polypropylene hydrophilic material, a non-woven polyethylene hydrophilic material, and/or a non-woven polyester hydrophilic material.

24. The fluid collection device of any of claim 20 or 21, wherein the outer layer includes a surface density of about 10 gsm to about 100 gsm.

25. The fluid collection device of any of claims 20-22, wherein the fluid permeable non-woven material of the inner layer includes a vertical non-woven foam material.

26. The fluid collection device of claim 23, wherein the vertical non-woven foam material includes one or more of polyester, nylon, polypropylene, polyethylene, and/or cellulose.

27. The fluid collection device of any of claims 20-24, wherein the inner layer includes a surface density of about 100 g/m²/cm to about 250 g/m²/cm.

28. The fluid collection device of any of claims 20-25, wherein the outer layer includes a thickness of about 0.125 mm to about 0.4 mm and the inner layer includes a thickness of about 2 mm to about 25 mm.

29. The fluid collection device of any of claims 20-26, wherein the adhesive includes a hotmelt adhesive including one or more of thermoplastic, polyurethane, acrylic, and/or polyester non-woven web adhesive.

30. The fluid collection device of any of claims 20-27, wherein the fluid impermeable barrier is substantially cylindrical, the fluid permeable body is substantially cylindrical, and the inner layer and the outer layer of the fluid permeable body are concentric with one another.

31. The fluid collection device of any of claims 20-28, further comprising a conduit extending through the aperture into chamber of the fluid impermeable barrier and at least partially into the fluid permeable body.

32. The fluid collection device of claim 29, wherein the conduit includes a shape memory material fixedly secured thereto and/or embedded therein.

33. The fluid collection device of claim 30, wherein the shape memory material includes one or more of a metal wire, a metal alloy wire, and/or a plastic reinforced fibers.
